(19) United States
(12) Patent Application Publication
(10) Pub. No.: US 2011/0004235 A1
(43) Pub. Date: Jan. 6, 2011

(54) TRANSAPICAL HEART PORT

(76) Inventors: Thoralf M. Sundt, III, Rochester, MN (US); Brent R. Phillips, Mukwonago, WI (US)

Correspondence Address: FISH & RICHARDSON P.C. (TC), PO BOX 1022, MINNEAPOLIS, MN, 55440-1022, US

(21) Appl. No.: 12/866,450

(22) PCT Filed: Feb. 5, 2009
(86) PCT No.: PCT/US09/33189

§ 371 Date: Sep. 10, 2010

Related U.S. Application Data
(60) Provisional application No. 61/027,291, filed on Feb. 8, 2008.

(51) Int. Cl. A61M 29/00

(52) U.S. Cl. 606/191

(57) ABSTRACT

This document relates to medical devices. For example, transapical heart ports, methods for making transapical heart ports, and methods for using transapical heart ports are provided.

### CROSS-REFERENCE TO RELATED APPLICATIONS

This application claims priority from U.S. Provisional Application Ser. No. 61/027,291, filed Feb. 8, 2008.

### BACKGROUND

1. Technical Field

This document relates to medical devices (e.g., transapical heart ports) and methods for using medical devices.

2. Background Information

Many cardiac surgical procedures require access to the interior of the heart. Transapical approaches to cardiac surgery can allow cardiac surgeons to access the interior of the heart via the apex. Through such access, a surgeon can, for example, replace or repair a mitral or aortic valve or can perform other surgical procedures.

### SUMMARY

This document relates to medical devices such as transapical heart ports. Such medical devices can be used to provide a cardiac surgeon with secure access to the interior of a heart. For example, a transapical heart port provided herein can allow cardiac surgeons to (1) perform heart surgeries with reduced tissue trauma and a reduced access site size, (2) induce less stress to the heart during initial access, (3) pass instruments in and out of the access site with ease, (4) maintain hemostasis particularly while passing instruments in and out of the access site, (5) gain access without the need of sutures, which can allow re-access to the heart without a risk of tearing sutures, and (6) obviate the need to remove the transapical heart port or repair the apex of the heart after the procedure (e.g., the transapical heart port may remain in place for an extended period of time or permanently).

In general, one aspect of this document features a transapical heart port that includes a housing having a first end and a second end, wherein the housing defines a channel extending between the first end and the second end, wherein the first end is configured to be inserted into a heart at the apex of the heart, wherein the channel is configured to provide repeated access to the interior of the heart through the channel, wherein the first end comprises a securing portion configured to secure the first end to an interior region of the heart, and wherein the second end comprises a securing portion configured to secure the second end to an exterior region of the heart, and a hemostatic valve attached to the housing and located within the channel, wherein the hemostatic valve is configured to reduce blood loss from the heart through the channel.

In some cases, the port can comprise pliable material that allows flexion of the port during beating of the heart. The size of the channel can allow passage of a prosthetic heart valve. The securing portion of the first end or the securing portion of the second end can comprise hooks configured to be embedded within the myocardium of the heart. The hooks can comprise a shape memory alloy for self-embedding within the myocardium upon deployment of the transapical heart port into the heart. The securing portion of the first end and the securing portion of the second end can comprise hooks configured to be embedded within the myocardium of the heart. The hooks can comprise a shape memory alloy for self-embedding within the myocardium upon deployment of the transapical heart port into the heart. The securing portion of the first end and/or the securing portion of the second end can comprise a balloon filled with a filler material that conforms to the contours of the heart. The filler material can be a liquid or gas. The filler material can harden over time. The filler material can remain pliable to allow flexion of the transapical heart port during beating of the heart. The balloon can comprise a donut shape configured about the first end or the second end. The balloon can comprise multiple flanges configured about the first end or the second end. The surface of the balloon can comprise a coating that promotes endothelization or cell growth. The securing portion of the first end can comprise a first balloon and the securing portion of the second end comprises a second balloon larger than the first balloon. The first and second balloons can be filled with a filler material that conforms to the contours of the heart. The second end can comprise multiple access points that allow multiple concurrent accesses. The transapical heart port can comprise a plug located within the channel and configured to provide permanent hemostasis. The plug can be secured to the channel using threads, snaps, hooks, or an adhesive. The plug can define a chamber configured to deliver drugs to the heart. The chamber can comprise an access site for refilling the chamber. The transapical heart port can comprise a sheath that provides access to the second end through the chest wall covering the heart. The sheath can be detachable from the transapical heart port. The sheath can comprise a hemostatic valve. The sheath can comprise two or more hemostatic valves. The sheath can define a balloon filler channel. The transapical heart port can comprise two or more hemostatic valves attached to the housing and located within the channel.

In another aspect, this document features a method for accessing the interior of a heart. The method comprises inserting a transapical heart port into the apex of the heart, securing the transapical heart port to the heart, and inserting an instrument into the heart through the transapical heart port. The transapical heart port can be a transapical heart port comprising: (a) a housing having a first end and a second end, wherein the housing defines a channel extending between the first end and the second end, wherein the first end is configured to be inserted into a heart at the apex of the heart, wherein the channel is configured to provide repeated access to the interior of the heart through the channel, wherein the first end comprises a securing portion configured to secure the first end to an interior region of the heart, and wherein the second end comprises a securing portion configured to secure the second end to an exterior region of the heart, and (b) a hemostatic valve attached to the housing and located within the channel, wherein the hemostatic valve is configured to reduce blood loss from the heart through the channel.

In some cases, the port can comprise pliable material that allows flexion of the port during beating of the heart. The size of the channel can allow passage of a prosthetic heart valve. The securing portion of the first end or the securing portion of the second end can comprise hooks configured to be embedded within the myocardium of the heart. The hooks can comprise a shape memory alloy for self-embedding within the myocardium upon deployment of the transapical heart port into the heart. The securing portion of the first end and the securing portion of the second end can comprise hooks configured to be embedded within the myocardium of the heart. The hooks can comprise a shape memory alloy for self-embedding within the myocardium upon deployment of the transapical heart port into the heart. The securing portion of the first end and/or the securing portion of the second end can comprise a balloon filled with a filler material that conforms to the contours of the heart. The filler material can be a liquid or gas. The filler material can harden over time. The filler material can remain pliable to allow flexion of the transapical heart port during beating of the heart. The balloon can comprise a donut shape configured about the first end or the second end. The balloon can comprise multiple flanges configured about the first end or the second end. The surface of the balloon can comprise a coating that promotes endothelization or cell growth. The securing portion of the first end can comprise a first balloon and the securing portion of the second end comprises a second balloon larger than the first balloon. The first and second balloons can be filled with a filler material that conforms to the contours of the heart. The second end can comprise multiple access points that allow multiple concurrent accesses. The transapical heart port can comprise a plug located within the channel and configured to provide permanent hemostasis. The plug can be secured to the channel using threads, snaps, hooks, or an adhesive. The plug can define a chamber configured to deliver drugs to the heart. The chamber can comprise an access site for refilling the chamber. The transapical heart port can comprise a sheath that provides access to the second end through the chest wall covering the heart. The sheath can be detachable from the transapical heart port. The sheath can comprise a hemostatic valve. The sheath can comprise two or more hemostatic valves. The sheath can define a balloon filler channel. The transapical heart port can comprise two or more hemostatic valves attached to the housing and located within the channel.

The securing can comprise embedding hooks into the myocardium of the heart. The hooks can self-embed using a shape memory alloy. The securing can comprise inflating at least one balloon. The inserting an instrument can comprise inserting a prosthetic heart valve into the heart. The inserting an instrument can comprise inserting a plug into the transapical heart port, wherein the plug delivers a drug to the heart. The inserting a transapical heart port can comprise inserting a sheath through the chest wall covering the heart and connecting the sheath to the transapical heart port. The inserting an instrument can comprise inserting the instrument through the sheath and the transapical heart port.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a cross-sectional view showing an example of a transapical heart port in a heart.

FIGS. 2A-2C are cross-sectional views showing examples of securing transapical heart ports in a heart.

FIGS. 3A-3B are top views showing examples of balloons for securing transapical heart ports.

FIG. 4 is a cross-sectional view showing an example of multiple access locations in a transapical heart port.

FIG. 5 is a cross-sectional view showing an example of a transapical heart port including a plug that provides long term hemostasis.

FIGS. 6A-6B are cross-sectional views showing examples of transapical heart ports having an attached sheath that provides access to the transapical heart port.

FIG. 7 is a flow chart showing an example of a process for accessing the interior of a heart.

Like reference symbols in the various drawings indicate like elements.

### DETAILED DESCRIPTION

This document relates to medical devices. For example, this document provides transapical heart ports, methods for making transapical heart ports, and methods for using transapical heart ports. The transapical heart ports provided herein can be inserted and secured to the apex of a beating heart to provide secure access to the inside or interior of the heart. The devices provided herein can have one or more one-way (or hemostatic) valves such that access to the inside of the heart via the apex is provided without blood loss around the instruments being introduced into the heart. The transapical heart ports provided herein can be used during surgeries where the patient's heart remains beating. The transapical heart ports provided herein can be used for inserting instruments of various types into the heart. For example, valves, catheters, suture devices, and repair devices can be inserted into a heart via a transapical heart port provided herein.

The transapical heart ports provided herein can be self-securing to the heart. For example, a transapical heart port provided herein can include a self-securing mechanism (e.g., a sutureless securing mechanism). In some cases, a transapical heart port provided herein can remain in place after completion of the operation being performed on the heart.

The transapical heart ports provided herein can have a deployment mechanism such as a dilator system over the wire. For example, a method such as the Seldinger technique used in cardiac catheterization labs can be used to deploy the transapical heart port. In some cases, a transapical heart port provided herein can be inserted at the apex of the heart, for example, using an open surgical incision or percutaneously. In some cases, a transapical heart port itself can provide secure access such that instruments can be exchanged during the intracavitary surgery without concern that one would lose control of the apex of the heart (e.g., to prevent bleeding through or around the transapical heart port and to maintain blood pressure in the patient).

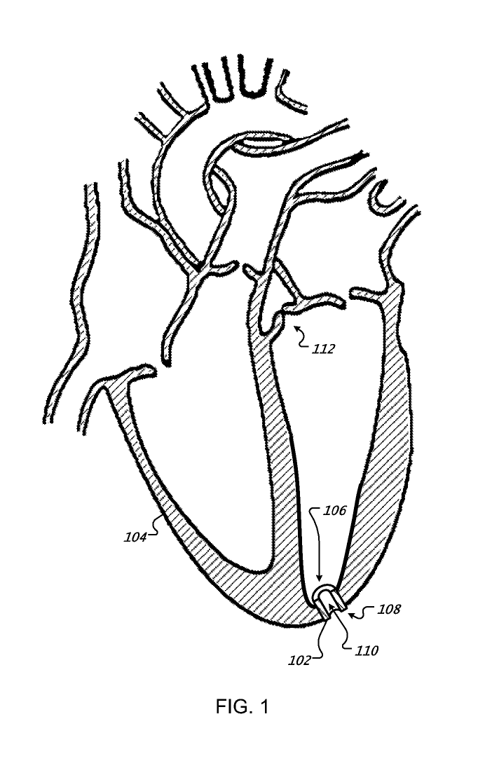

FIG. 1 is a cross-sectional view showing an example of a transapical heart port 102 in a heart 104. The transapical heart port 102 includes a housing having a first end 106. The first end 106 is inserted into the heart 104 at the apex of the heart 104. While shown here as being inserted in the left ventricle of the heart 104, the transapical heart port 102 can also be inserted into the right ventricle of the heart 104. The housing of the transapical heart port 102 also includes a second end 108. The housing with first end 106 and the second end 108 defines a channel 110. The channel 110 provides access to the interior of the heart 104. For example, a drug can be delivered to the interior of the heart 104 through the channel 110. In some cases, a heart valve 112 can be repaired or replaced via access through the channel 110.

The transapical heart port 102 can be made of various materials, such as metals, plastics, and polymers. In some cases, the transapical heart port 102 can be made of a material that is pliable. The pliable material may allow flexion of the transapical heart port 102 during beating of the heart 104. The flexion of the material in the transapical heart port 102 can prevent or reduce tissue damage to the heart 104 and dislodging of the transapical heart port 102 from the heart 104.

In some cases, the channel 110 has an interior diameter that is sufficiently large to allow passage of a prosthetic heart valve through the channel 110. For example, the channel 110 may have a diameter of five or more millimeters (e.g., at least five, six, seven, eight, nine, ten, eleven, twelve, 13, 14, 15, 16, 17, 18, 19, or 20 mm). In some cases, the diameter can be between about 10 mm and about 12 mm. A prosthetic heart valve may be used, for example, in replacing the heart valve 112.

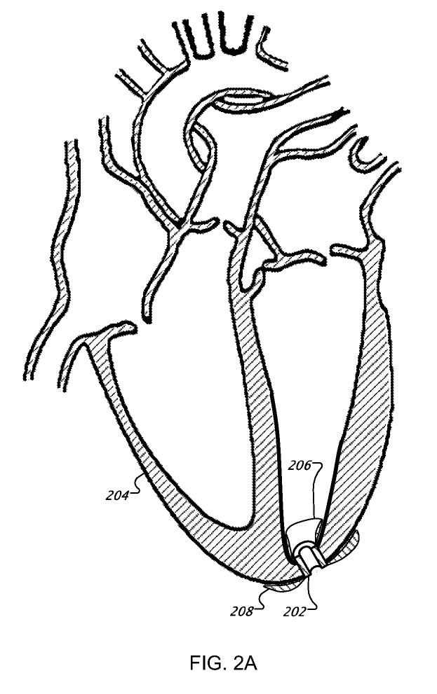

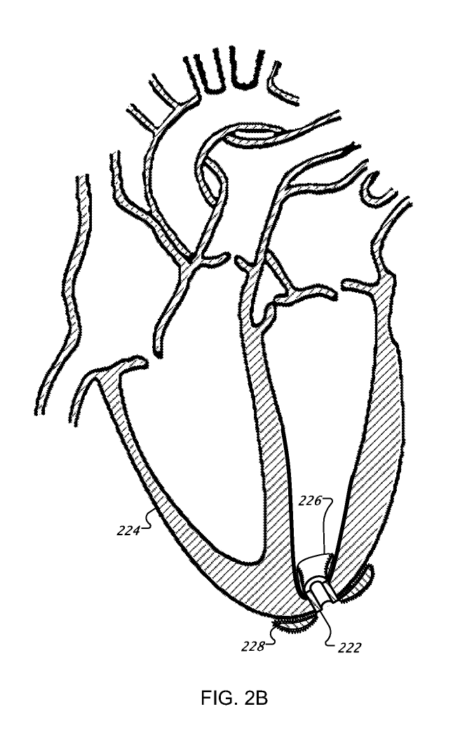

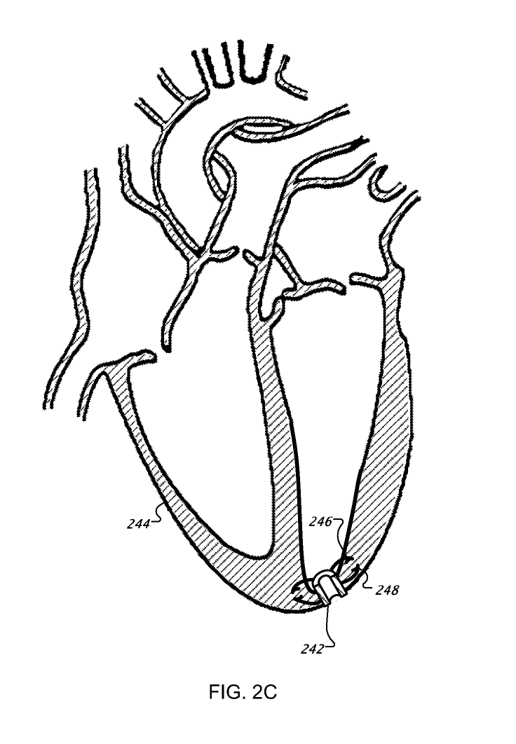

FIGS. 2A-2C are cross-sectional views showing examples of securing transapical heart ports in a heart. In some cases, the method used to secure the transapical heart port provides a pull-out strength with a pressure of between about 300 and about 400 mm of mercury (mmHg). For example, the method used to secure the transapical heart port can provide a pull-out strength with a pressure of at least 300 mmHg. FIG. 2A shows a transapical heart port 202 in a heart 204. The transapical heart port 202 is secured at the interior of the heart 204 by one or more interior balloons 206. The transapical heart port 202 is also secured at the exterior of the heart 204 by one or more exterior balloons 208. In some cases, the exterior balloons 208 cover a greater surface area of the heart 204 than the interior balloons 206. The balloons can be made using biomedical balloon material.

The interior balloons 206 and the exterior balloons 208 can secure the transapical heart port 202 to the heart 204 by conforming to the anatomy of the heart 204. For example, the balloons can conform to the curvature of the interior and exterior heart walls as well as thickness of the heart wall at the apex of the heart 204.

In some cases, the interior balloons 206 and/or the exterior balloons 208 are filled or inflated with a gas (e.g., carbon dioxide) or a liquid (e.g., saline). In some case, the material used to fill the balloons can harden over time. For example, a polymer such as acrylate, foam, or gel can be used to fill the balloons. In some cases, the hardness of the filler material remains pliable to allow flexion during beating of the heart 204.

FIG. 2B shows a transapical heart port 222 in a heart 224. The transapical heart port 222 is secured at the interior of the heart 224 by one or more interior balloons 226. The transapical heart port 222 is secured at the exterior of the heart 224 by one or more exterior balloons 228. In some cases, the interior balloons 226 and/or the exterior balloons 228 can have a surface that promotes cell growth or endothelization. The surface also can limit thrombolytic potential. In some cases, the surface can further secure the transapical heart port 222 to the heart 224. For example, the transapical heart port 222 and/or the balloons can be coated with a material that promotes cell growth. In another example, the transapical heart port 222 and/or the balloons can have a surface texture that promotes cell growth and/or further secures the transapical heart port 222 to the heart 224. In some cases, the balloons can be made of (or coated) with a fabric, such as Dacron. In some cases, the balloons can be coated with a cell growth compound such as collagen, fibrin, or another cell growth promoting biomatrix.

FIG. 2C shows a transapical heart port 242 in a heart 244. The transapical heart port 242 can include one or more interior hooks 246 and one or more exterior hooks 248 that secure the transapical heart port 242 to the heart 244. Particularly, the hooks can embed within the myocardium of the heart 244. In some cases, the tips of the hooks have barbs that hold the hooks in the myocardium. In some cases, the hooks are deployed by an active process, such as by actuating a hinge of a hook. In some cases, the hooks self-embed within the myocardium. For example, the hooks can include a shape memory alloy, such as nickel titanium or nitinol. Deploying the hooks in the heart 244 can warm the shape memory alloy material in the hooks, causing the hooks to reshape or bend and embed within the myocardium of the heart 244.

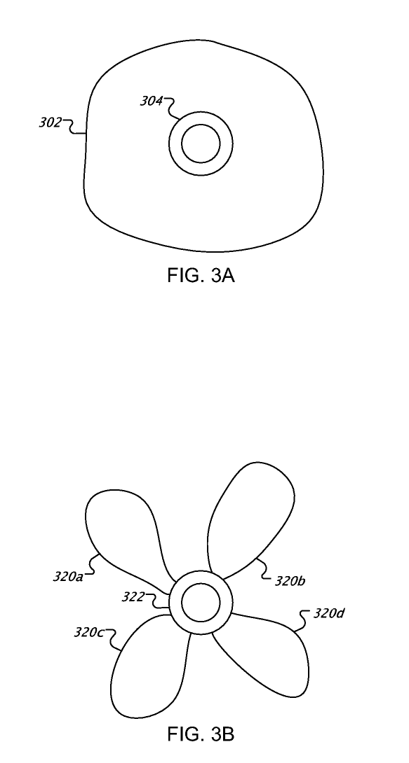

FIGS. 3A-3B are top views showing examples of balloons for securing transapical heart ports. FIG. 3A shows a circular balloon 302. The circular balloon 302 forms a round or doughnut shape about the opening of a transapical heart port 304. FIG. 3B shows multiple flange balloons 320a-d. The flange balloons 320a-d can be distributed about the opening of a transapical heart port 322. The circular balloon 302 and the flange balloons 320a-d can act to secure a transapical heart port to a heart as described herein.

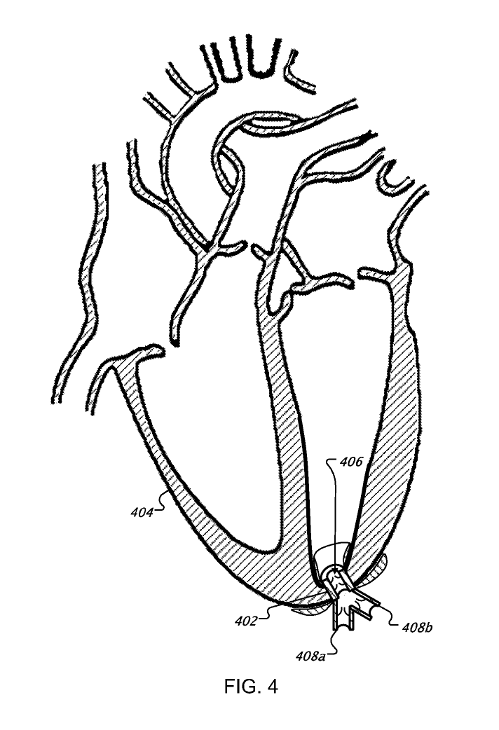

FIG. 4 is a cross-sectional view showing an example of multiple access locations in a transapical heart port 402. The transapical heart port 402 is inserted in a heart 404. The transapical heart port 402 includes one or more hemostatic valves 406. The hemostatic valves can prevent or reduce blood loss from the heart 404. For example, the hemostatic valves 406 can include one-way valves and/or a self-sealing septum as is described in U.S. Pat. No. 5,718,682, filed on Jun. 28, 1996, by Elton M. Tucker, and entitled “Access port device and method of manufacture.”

The transapical heart port 402 also includes multiple access points 408a-b. The access points 408a-b can provide for multiple concurrent accesses to the interior of the heart 404 through the transapical heart port 402. For example, multiple instruments can be inserted into the heart 404 simultaneously through the transapical heart port 402 using the access points 408a-b. In some cases, the access points 408a-b include one or more hemostatic valves that prevent or reduce blood loss from the heart 404.

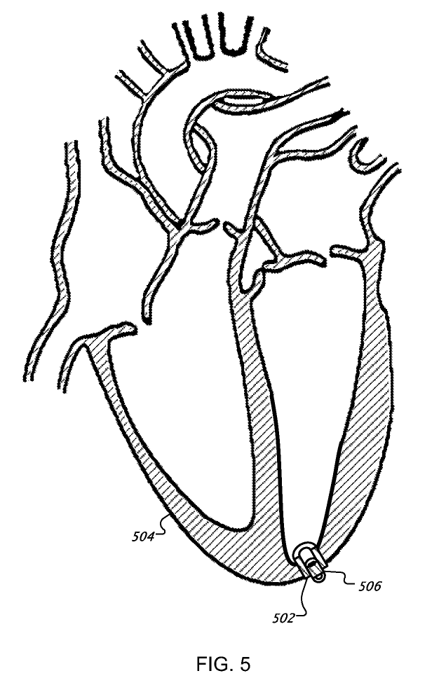

FIG. 5 is a cross-sectional view showing an example of a transapical heart port 502 in a heart 504. The transapical heart port 502 can include a plug 506 that provides long term or permanent hemostasis. The plug 506 can be left in place within the transapical heart port 502 over an extended period of time. The plug 506 can engage the transapical heart port 502 using, for example, snaps, hooks, adhesive (e.g., glue), or other securing methods. In some cases, the plug 506 can be removed for subsequent procedures that access the interior of the heart 504. In one example, a system can employ threads within the transapical heart port 502 and around the plug 506. A tool can be used to screw and unscrew the plug 506. In some cases, the plug 506 can have a pull-out strength with a pressure between about 500 and about 600 mmHg. For example, the plug 506 can have a pull-out strength with a pressure greater than about 300 mmHg. In some cases, the plug 506 can include a chamber for delivering one or more drugs to the interior of the heart 504. Such drugs can be released over time. In some cases, the plug 506 can include an access site for re-filling the drug chamber.

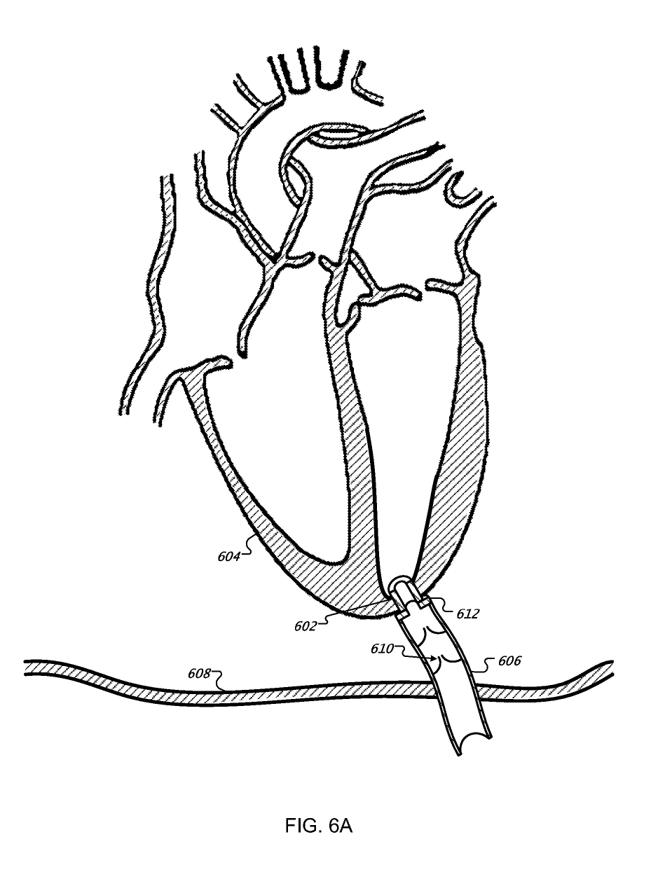

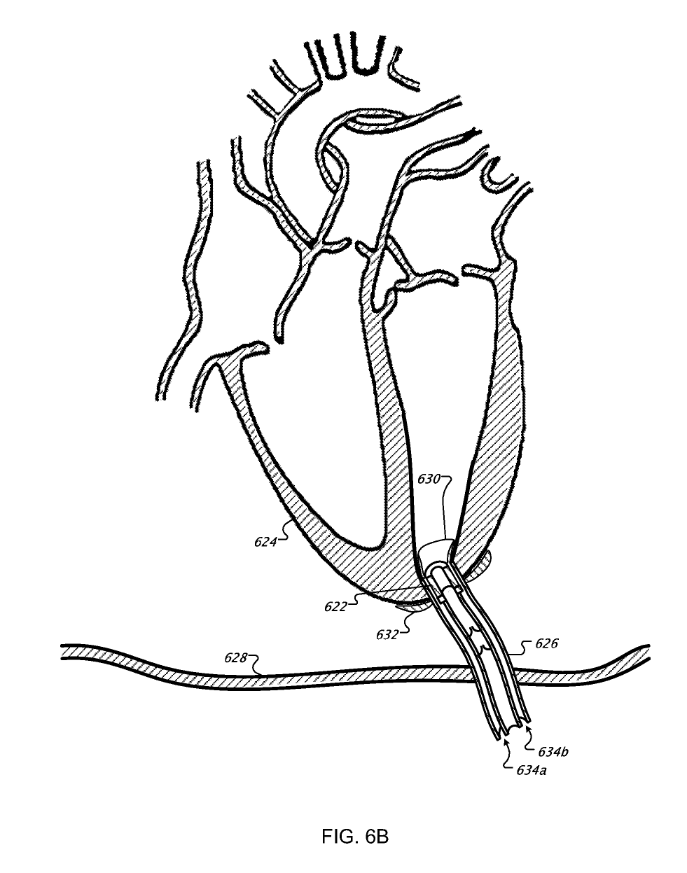

FIG. 6A is a cross-sectional view showing an example of a transapical heart port 602 in a heart 604. Attached to the transapical heart port 602 is a sheath 606 that provides access to the transapical heart port 602 through a chest wall 608 covering the heart 604. In some cases, the sheath 606 can include one or more hemostatic valves 610 that prevent or reduce blood loss from the heart 604. The sheath 606 can provide access to the transapical heart port 602 (and correspondingly the interior of the heart 604) from outside the body through the chest wall 608. While described herein as passing through the “chest wall,” the sheath 606 can pass through an alternate part of the body, such as the abdomen or neck. In some cases, the sheath 606 can prevent or reduce the need for large surgical incisions and/or damage to intervening tissue that results from passing surgical instruments through the body to the transapical heart port 602. In some cases, the sheath 606 can be detached from the transapical heart port 602 after completion of a procedure that accesses the interior of the heart 604. In some cases, a plug, such as the plug 506 described with respect to FIG. 5, can be passed and installed in the transapical heart port 602 through the sheath 606.

The sheath 606 can include an attachment device 612, such as a threaded screw, clips, or luer locks. The sheath 606 can be attached to the transapical heart port 602 prior to inserting the transapical heart port 602 into the heart 604 or after inserting the transapical heart port 602 into the heart 604. The sheath 606 can be detached from the transapical heart port 602 after performing a procedure and a sheath can be reattached for another procedure at a later time.

FIG. 6B is a cross-sectional view showing an example of a transapical heart port 622 in a heart 624. Attached to the transapical heart port 622 is a sheath 626 that provides access to the transapical heart port 622 through a chest wall 628. The transapical heart port 622 can be secured to the heart 624 by one or more interior balloons 630 and one or more exterior balloons 632. The sheath 626 can include an inner wall and an outer wall that define one or more inflation channels 634a-b. The inflation channels 634a-b can provide access to the balloons for filling the balloons with a gas or liquid. In some cases, a plug, such as the plug 506 described with respect to FIG. 5, can be inserted into the transapical heart port 622 through the sheath 626. The plug can be used to sever and seal the inflation ports that connect the sheath 626 to the balloons.

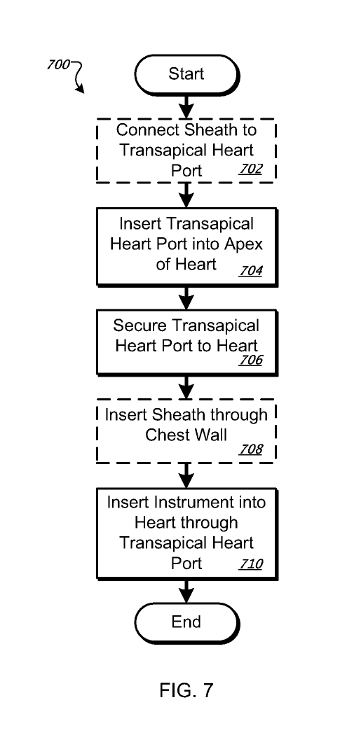

FIG. 7 is a flow chart showing an example of a process 700 for accessing the interior of a heart. The process 700 can be performed, for example, by a device such as the transapical heart ports 102, 202, 222, 242, 302, 322, 402, 502, 602, and 622. For clarity of presentation, the description that follows uses the transapical heart ports 102, 202, 222, 242, 302, 322, 402, 502, 602, and 622 as the basis of an example for describing the process 700. However, another device, or combination of devices, can be used to perform the process 700. Optionally, the process 700 begins with connecting (702) a sheath to a transapical heart port. For example, the sheath 626 can be connected to the transapical heart port 622. The process 700 inserts (704) the transapical heart port into the apex of a heart. For example, the transapical heart port 102 can be inserted into the apex of the heart 104 through an incision or percutaneously. A ventricle, such as the left ventricle, can be accessed with a wire (e.g., small theracotomy).

In some implementations, the Seldinger technique may be used to insert the transapical heart port into the apex of the heart. A needle is inserted into the apex of the heart. A guide wire can be advanced through the needle and into the interior of the heart. The needle is then removed, and the guide wire is left in place. The guide wire is used to insert a dialator/introducer system. The transapical heart port (and optionally the sheath) can be inserted on the introducer system over the wire. After the transapical heart port is inserted, the guide wire and introducer system can be removed.

The process 700 secures (706) the transapical heart port to the heart. For example, the transapical heart ports 302 and 322 can be secured using balloons while the transapical heart port 342 can be secured using hooks. In some cases, a combination of interior and/or exterior securing devices can be used. In some cases, a combination of balloons and hooks can be used. In some implementations, the balloons attached to the transapical heart port 622 can be filled from outside the body through the inflation channels 634a-b in the sheath 626.

Optionally, the process 700 inserts (708) a sheath through a chest wall that covers the heart. For example, the sheath 626 can be inserted through the chest wall 628.

The process 700 inserts (710) an instrument into the heart through the transapical heart port. For example, a prosthetic valve or a plug that delivers a drug to the heart can be inserted through the transapical heart port 622. In some cases, the insertion can occur from outside the body through the sheath 626. In some implementations, the sheath can be detached from the transapical heart port after the procedure. In some implementations, the transapical heart port remains in place after removal of the sheath. In some implementations, inserting the plug into the transapical heart port detaches the sheath from the transapical heart port. The connection between the sheath and the transapical heart port can be made using threads (e.g., screwing a portion of the sheath onto or into a portion of the transapical heart port), clips, luer locks, etc.

Although a few implementations have been described in detail above, other modifications are possible. For example, the logic flows depicted in the figures do not require the particular order shown, or sequential order, to achieve desirable results. In addition, other steps may be provided, or steps may be eliminated, from the described flows, and other components may be added to, or removed from, the described systems and devices. Accordingly, other implementations are within the scope of the following claims.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A transapical heart port comprising:
a housing having a first end and a second end, wherein said housing defines a channel extending between said first end and said second end, wherein said first end is configured to be inserted into a heart at the apex of said heart, wherein said channel is configured to provide repeated access to the interior of said heart through said channel, wherein said first end comprises a securing portion configured to secure said first end to an interior region of said heart, and wherein said second end comprises a securing portion configured to secure said second end to an exterior region of said heart, and
a hemostatic valve attached to said housing and located within said channel, wherein said hemostatic valve is configured to reduce blood loss from said heart through said channel.

2. The transapical heart port of claim 1, wherein said port comprises pliable material that allows flexion of said port during beating of said heart.

3. The transapical heart port of claim 1, wherein the size of said channel allows passage of a prosthetic heart valve.

4. The transapical heart port of claim 1, wherein said securing portion of said first end or said securing portion of said second end comprises hooks configured to be embedded within the myocardium of said heart.

5. The transapical heart port of claim 4, wherein said hooks comprise a shape memory alloy for self-embedding within the myocardium upon deployment of said transapical heart port into said heart.

6. The transapical heart port of claim 1, wherein said securing portion of said first end and said securing portion of said second end comprise hooks configured to be embedded within the myocardium of said heart.

7. The transapical heart port of claim 6, wherein said hooks comprise a shape memory alloy for self-embedding within the myocardium upon deployment of said transapical heart port into said heart.

8. The transapical heart port of claim 1, wherein said securing portion of said first end and/or said securing portion of said second end comprises a balloon filled with a filler material that conforms to the contours of said heart.

9. The transapical heart port of claim 8, wherein said filler material is a liquid or gas.

10. The transapical heart port of claim 8, wherein said filler material hardens over time.

11. The transapical heart port of claim 8, wherein said filler material remains pliable to allow flexion of said transapical heart port during beating of said heart.

12. The transapical heart port of claim 8, wherein said balloon comprises a donut shape configured about said first end or said second end.

13. The transapical heart port of claim 8, wherein said balloon comprises multiple flanges configured about said first end or said second end.

14. The transapical heart port of claim 8, wherein the surface of said balloon comprises a coating that promotes endothelization or cell growth.

15. The transapical heart port of claim 8, wherein said securing portion of said first end comprises a first balloon and said securing portion of said second end comprises a second balloon larger than said first balloon.

16. The transapical heart port of claim 15, wherein said first and second balloons are filled with a filler material that conforms to the contours of said heart.

17. The transapical heart port of claim 1, wherein said second end comprises multiple access points that allow multiple concurrent accesses.

18. The transapical heart port of claim 1, wherein said transapical heart port comprises a plug located within the channel and configured to provide permanent hemostasis.

19. The transapical heart port of claim 18, wherein said plug is secured to said channel using threads, snaps, hooks, or an adhesive.

20. The transapical heart port of claim 18, wherein said plug defines a chamber configured to deliver drugs to said heart.

21. The transapical heart port of claim 20, wherein said chamber comprises an access site for refilling said chamber.

22. The transapical heart port of claim 1, wherein said transapical heart port comprises a sheath that provides access to said second end through the chest wall covering said heart.

23. The transapical heart port of claim 22, wherein said sheath is detachable from said transapical heart port.

24. The transapical heart port of claim 22, wherein said sheath comprises a hemostatic valve.

25. The transapical heart port of claim 22, wherein said sheath comprises two or more hemostatic valves.

26. The transapical heart port of claim 22, wherein said sheath defines a balloon filler channel.

27. The transapical heart port of claim 1, wherein said transapical heart port comprises two or more hemostatic valves attached to said housing and located within said channel.

28. A method for accessing the interior of a heart, wherein said method comprises inserting a transapical heart port into the apex of said heart, securing said transapical heart port to said heart, and inserting an instrument into said heart through said transapical heart port.

29. The method of claim 28, wherein said transapical heart port is the transapical heart port of any one of claims 1-27.

30. The method of claim 28, wherein said securing comprises embedding hooks into the myocardium of said heart.

31. The method of claim 30, wherein said hooks self-embed using a shape memory alloy.

32. The method of claim 28, wherein said securing comprises inflating at least one balloon.

33. The method of claim 28, wherein said inserting an instrument comprises inserting a prosthetic heart valve into said heart.

34. The method of claim 28, wherein said inserting an instrument comprises inserting a plug into said transapical heart port, wherein said plug delivers a drug to said heart.

35. The method of claim 28, wherein said inserting a transapical heart port comprises inserting a sheath through the chest wall covering said heart and connecting said sheath to said transapical heart port.

36. The method of claim 35, wherein said inserting an instrument comprises inserting said instrument through said sheath and said transapical heart port.
